(19) United States
(12) Patent Application Publication
(10) Pub. No.: US 2006/0200025 A1
(43) Pub. Date: Sep. 7, 2006

(54) Systems, methods, and apparatus for automatic software flow using instrument detection during computer-aided surgery

(76) Inventors: Scott Elliott, Terey Hills (AU); Daniel McCombs, Memphis, TN (US)

Correspondence Address: CHIEF PATENT COUNSEL;SMITH & NEPHEW, INC., 1450 BROOKS ROAD, MEMPHIS, TN, 38116, US

(21) Appl. No.: 11/296,851

(22) Filed: Dec. 1, 2005

Related U.S. Application Data
(60) Provisional application No. 60/632,628, filed on Dec. 2, 2004.

(51) Int. Cl. A61B 5/05

(52) U.S. Cl. 600/424

(57) ABSTRACT

Systems, methods, and apparatuses for automatic software flow using instrument detection during a computer-aided surgery. At least system in accordance with an embodiment of the invention includes a computer-aided surgical navigational system with a display screen and at least one sensor. The system can include a processor capable of detecting a plurality of arrays, wherein each array is associated with a respective surgical instrument. The processor is further capable of determining a respective surgical procedure associated with the respective surgical instrument, based at least in part on detecting at least one array using the sensor. In addition, the processor is capable of outputting via the screen at least one user interface associated with the respective surgical procedure associated with the respective surgical instrument.

### RELATED APPLICATIONS

This application claims priority to U.S. Provisional Ser. No. 60/632,628, entitled “Automatic Software Flow Using Instrument Detection,” filed on Dec. 2, 2004, which is incorporated by reference.

### FIELD OF THE INVENTION

The invention relates generally to systems, methods, and apparatus related to computer aided-surgery, and more specifically to systems, methods, and apparatus for automatic software flow using instrument detection during a computer-aided surgery.

### BACKGROUND OF THE INVENTION

Many surgical procedures require a wide array of instrumentation and other surgical items. Such items may include, but are not limited to: sleeves to serve as entry tools, working channels, drill guides and tissue protectors; scalpels; entry awls; guide pins; reamers; reducers; distractors; guide rods; endoscopes; arthroscopes; saws; drills; screwdrivers; awls; taps; osteotomes, wrenches, trial implants and cutting guides. In many surgical procedures, including orthopedic procedures, it may be desirable to associate some or all of these items with a guide and/or handle incorporating a navigational reference, allowing the instrument to be used with a computer-aided surgical navigation system.

Several manufacturers currently produce computer-aided surgical navigation systems. The TREON™ and ION™ systems with FLUORONAV™ software manufactured by Medtronic Surgical Navigation Technologies, Inc. are examples of such systems. The BrainLAB VECTORVISION™ system is another example of such a surgical navigation system. Systems and processes for accomplishing computer-aided surgery are also disclosed in U.S. Ser. No. 10/084,012, filed Feb. 27, 2002 and entitled “Total Knee Arthroplasty Systems and Processes”; U.S. Ser. No. 10/084,278, filed Feb. 27, 2002 and entitled “Surgical Navigation Systems and Processes for Unicompartmental Knee Arthroplasty”; U.S. Ser. No. 10/084,291, filed Feb. 27, 2002 and entitled “Surgical Navigation Systems and Processes for High Tibial Osteotomy”; International Application No. US02/05955, filed Feb. 27, 2002 and entitled “Total Knee Arthroplasty Systems and Processes”; International Application No. US02/05956, filed Feb. 27, 2002 and entitled “Surgical Navigation Systems and Processes for Unicompartmental Knee Arthroplasty”; International Application No. US02/05783 entitled “Surgical Navigation Systems and Processes for High Tibial Osteotomy”; U.S. Ser. No. 10/364,859, filed Feb. 11, 2003 and entitled “Image Guided Fracture Reduction,” which claims priority to U.S. Ser. No. 60/355,886, filed Feb. 11, 2002 and entitled “Image Guided Fracture Reduction”; U.S. Ser. No. 60/271,818, filed Feb. 27, 2001 and entitled “Image Guided System for Arthroplasty”; and U.S. Ser. No. 10/229,372, filed Aug. 27, 2002 and entitled “Image Computer Assisted Knee Arthroplasty”, the entire contents of each of which are incorporated herein by reference as are all documents incorporated by reference therein.

These systems and processes use position and/or orientation tracking sensors such as infrared sensors acting stereoscopically or other sensors acting in conjunction with navigational references to track positions of body parts, surgery-related items such as implements, instrumentation, trial prosthetics, prosthetic components, and virtual constructs or references such as rotational axes which have been calculated and stored based on designation of bone landmarks. Sensors, such as cameras, detectors, and other similar devices, are typically mounted overhead with respect to body parts and surgery-related items to receive, sense, or otherwise detect positions and/or orientations of the body parts and surgery-related items. Processing capability such as any desired form of computer functionality, whether standalone, networked, or otherwise, takes into account the position and orientation information as to various items in the position sensing field (which may correspond generally or specifically to all or portions or more than all of the surgical field) based on sensed position and orientation of their associated navigational references, or based on stored position and/or orientation information. The processing functionality correlates this position and orientation information for each object with stored information, such as a computerized fluoroscopic imaged file, a wire frame data file for rendering a representation of an instrument component, trial prosthesis or actual prosthesis, or a computer generated file relating to a reference, mechanical, rotational or other axis or other virtual construct or reference. The processing functionality then displays position and orientation of these objects on a rendering functionality, such as a screen, monitor, or otherwise, in combination with image information or navigational information such as a reference, mechanical, rotational or other axis or other virtual construct or reference. Thus, these systems or processes, by sensing the position of navigational references, can display or otherwise output useful data relating to predicted or actual position and orientation of surgical instruments, body parts, surgically related items, implants, and virtual constructs for use in navigation, assessment, and otherwise performing surgery or other operations.

Some of the navigational references used in these systems may emit or reflect infrared light that is then detected by infrared sensors. The references may be sensed actively or passively by infrared, visual, sound, magnetic, electromagnetic, x-ray or any other desired technique. An active reference emits energy, and a passive reference merely reflects energy. Some navigational references may have markers or fiducials that are traced by an infrared sensor to determine the position and orientation of the reference and thus the position and orientation of the associated instrument, item, implant component or other object to which the reference is attached.

In addition to navigational references with fixed fiducials, modular fiducials, which may be positioned independent of each other, may be used to reference points in the coordinate system. Modular fiducials may include reflective elements which may be tracked by two, sometimes more, sensors whose output may be processed in concert by associated processing functionality to geometrically calculate the position and orientation of the item to which the modular fiducial is attached. Like fixed fiducial navigational references, modular fiducials and the sensors need not be confined to the infrared spectrum-any electromagnetic, electrostatic, light, sound, radio frequency or other desired technique may be used. Similarly, modular fiducials may “actively” transmit reference information to a tracking system, as opposed to “passively” reflecting infrared or other forms of energy.

Navigational references useable with the above-identified navigation systems may be secured to any desired structure, including the above-mentioned surgical instruments and other items. The navigational references may be secured directly to the instrument or item to be referenced. However, in many instances it will not be practical or desirable to secure the navigational references to the instrument or other item. Rather, in many circumstances it will be preferred to secure the navigational references to a handle and/or a guide adapted to receive the instrument or other item. For example, drill bits and other rotating instruments cannot be tracked by securing the navigational reference directly to the rotating instrument because the reference would rotate along with the instrument. Rather, a preferred method for tracking a rotating instrument is to associate the navigational reference with the instrument or item's guide or handle.

Some or all of the computer-aided surgical navigation systems disclosed above can be used in conjunction with various surgeries to provide surgical-related information during surgery. For example, some computer-aided surgical navigation systems can include a display screen with a series of user interfaces to provide surgical-related information during a particular surgery. The display screen and user interfaces can provide particular information associated with a surgical procedure being performed, and can also display visual representations of surgery-related items such as instrumentation which may be utilized during the surgical procedure. However, in some instances during a computer-aided surgery, a user such as a surgeon or other surgical personnel must press buttons or foot pedals associated with the computer-aided surgical navigation system to scroll or otherwise navigate through the user interfaces on the display screen. Associated software may receive the user inputs and corresponding display user interfaces in accordance with the user inputs. This type of user interaction with the computer-aided surgical navigation system can be time consuming. In some instances, if an incorrect input or command is entered by the user, the user must then scroll or navigate backwards through the user interfaces and re-enter a correct input or command, thereby adding time to the surgical procedure. In other instances, if a user desires to deviate from a pre-defined set of steps associated with the user interfaces on the display screen, the user must scroll or navigate through the user interfaces, or otherwise manually input a desired surgical procedure to obtain a desired user interface, thereby adding time to the surgical procedure.

### SUMMARY OF THE INVENTION

Systems and methods according to various embodiments of the invention address some or all of the above issues and combinations thereof. They do so by providing a computer-aided surgical system, methods and surgical methods, and apparatus for providing automatic software flow using instrument detection during a surgical procedure involving an orthopedic implant device, a bone, and/or bone implant or structure. During a computer-aided surgery, the computer-aided surgical system and methods can automatically provide a user interface associated with a surgical procedure for a user such as a surgeon or other surgical personnel. Such systems and methods are particularly useful for surgeons installing orthopedic components within a patient's body, wherein the computer-aided surgical navigation system can automatically display a user interface associated with a surgical procedure of interest when a particular surgical instrument, position of the instrument, or proximity or position of the instrument relative to a patient's body is detected or otherwise identified by the system.

One aspect of systems, methods, and apparatuses according to various embodiments of the invention, focuses on computer-aided surgical navigational system with a display screen and at least one sensor. The system can include a processor capable of detecting a plurality of arrays using the sensor, wherein each array is associated with a respective surgical instrument. The processor is further capable of determining a respective surgical procedure associated with the respective surgical instrument, based at least in part on detecting at least one array. In addition, the processor is capable of outputting via the screen at least one user interface associated with the respective surgical procedure associated with the respective surgical instrument.

According to another aspect of the invention, systems, methods, and apparatuses according to various embodiments of the invention include a method performed by a computer-aided surgical navigational system with a display screen and at least one sensor. The method can include associating a plurality of arrays with a plurality of surgical instruments, wherein each array is associated with a respective surgical instrument. In addition, the method can include associating the plurality of surgical instruments with a plurality of surgical procedures, wherein each surgical instrument is associated with a respective surgical procedure. Furthermore, the method can include associating the plurality of surgical procedures with a plurality of user interfaces, wherein each surgical procedure is associated with at least one respective user interface. Moreover, the method can include detecting at least one array. The method can also include based at least in part on detecting the array using the sensor, determining a respective surgical procedure associated with a respective surgical instrument. Further, the method can include outputting via the screen at least one user interface associated with the respective surgical procedure associated with the respective surgical instrument.

According to yet another aspect of the invention, systems, methods, and apparatuses according to various embodiments of the invention can include a computer-aided surgical navigational system with a display screen and at least one sensor. The system can include a probe capable of contacting a portion of a plurality of arrays associated with a plurality of surgical instruments, wherein each array is associated with a respective surgical instrument. In addition, the system can include a processor capable of detecting the contacted portion of at least one array associated with a respective surgical instrument. The processor can also be capable of determining a respective surgical procedure associated with the respective surgical instrument, based at least in part on detection of the contacted portion of the array associated with a respective surgical instrument using the sensor. The processor is further capable of outputting via the screen at least one user interface associated with the respective surgical procedure associated with the respective surgical instrument.

According to yet another aspect of the invention, systems, methods, and apparatuses according to various embodiments of the invention can include a method performed by a computer-aided surgical navigational system with a display screen and at least one sensor. The method can include associating a plurality of arrays with a plurality of surgical instruments, wherein each array is associated with a respective surgical instrument. In addition, the method can include associating the plurality of surgical instruments with a plurality of surgical procedures, wherein each surgical instrument is associated with a respective surgical procedure. Furthermore, the method can include associating the plurality of surgical procedures with a plurality of user interfaces, wherein each surgical procedure is associated with at least one respective user interface. Furthermore, the method can include detecting a portion of the array that has been contacted with a probe. The method can also include determining a respective surgical procedure associated with a respective surgical instrument, based at least in part on detecting the contacted portion of the array using the sensor. Moreover, the method can include outputting via the screen at least one user interface associated with the respective surgical procedure associated with the respective surgical instrument.

According to yet another aspect of the invention, systems, methods, and apparatuses according to various embodiments of the invention can include a computer-aided surgical navigational system with a display screen and a sensor. The system can include a processor capable of detecting an array associated with a portion of a patient's body. In addition, the processor is capable of detecting a plurality of arrays associated with plurality of surgical instruments using the sensor, wherein each array is associated with a respective surgical instrument. Furthermore, the processor is capable of determining a position of at least one array associated with a respective surgical instrument. Moreover, the processor is capable of determining a respective surgical procedure associated with the position of a particular array associated with the respective surgical instrument, based at least in part on determining the position of the array with respect to the portion of the patient's body using the sensor. Furthermore, the processor is capable of outputting via the screen at least one user interface associated with a respective surgical procedure associated with the respective surgical instrument.

According to yet another aspect of the invention, systems, methods, and apparatuses according to various embodiments of the invention can include a method performed by a computer-aided surgical navigational system with a display screen and at least one sensor. The method can also include associating a plurality of arrays with a plurality of surgical instruments and a portion of a patient's body, wherein each array is associated with a respective surgical instrument or a portion of the patient's body. In addition, the method can include associating the plurality of surgical instruments with a plurality of surgical procedures, wherein each surgical instrument is associated with a respective surgical procedure. Further, the method can include associating the plurality of surgical procedures with a plurality of user interfaces, wherein each surgical procedure is associated with at least one user interface. The method can also include detecting at least one array associated with a portion of the patient's body. In addition, the method can include detecting at least one array associated with a surgical instrument. Moreover, the method can include determining a respective surgical procedure associated with a respective surgical instrument, based at least in part on the position of the array associated with a portion of the patient's body relative to the array associated with a surgical instrument using the sensor. The method can also include outputting via the screen at least one user interface associated with the respective surgical procedure associated with the respective surgical instrument.

According to yet another aspect of the invention, systems, methods, and apparatuses according to various embodiments of the invention can include a surgical method performed in conjunction with a computer-aided surgical navigational system with a display screen and at least one sensor. The surgical method can include manipulating a surgical instrument associated with an array, wherein the array can be detected by the at least one sensor. The surgical method can also include based at least in part on manipulating the particular array, receiving via the screen at least one user interface associated with a respective surgical procedure associated with the respective surgical instrument.

According to yet another aspect of the invention, systems, methods, and apparatuses according to various embodiments of the invention can include a surgical method performed in conjunction with a computer-aided surgical navigational system with a display screen and at least one sensor. The surgical method can include manipulating a surgical instrument associated with an array, wherein the array can be detected by the at least one sensor. In addition, the surgical method can include contacting a probe with a portion of the array associated with the surgical instrument, wherein the contact of the probe with the array can be detected by the at least one sensor. Furthermore, the surgical method can include based at least in part on detecting the contact of the probe with the array, receiving via the screen at least one user interface associated with a respective surgical procedure associated with the respective surgical instrument.

According to yet another aspect of the invention, systems, methods, and apparatuses according to various embodiments of the invention can include a surgical method performed in conjunction with a computer-aided surgical navigational system with a display screen and at least one sensor. The surgical method can include manipulating a portion of a patient's body associated with a first array, wherein the first array can be detected by the at least one sensor. In addition, the surgical method can include manipulating a surgical instrument associated with a second array relative to the portion of the patient's body, wherein the second array can be detected by the at least one sensor. Furthermore, the surgical method can include based at least in part on the position of the surgical instrument relative to the portion of the patient's body, receiving via the screen at least one user interface associated with a respective surgical procedure associated with the respective surgical instrument.

Objects, features and advantages of various systems, methods, and apparatuses according to various embodiments of the invention include:

(1) providing the ability to automate software flow using instrument detection during a computer-aided surgery;

(2) providing the ability to automate software flow in a computer-aided navigation system using instrument detection during a computer-aided surgical procedure;

(3) providing the ability for a user to manipulate a surgical instrument during a computer-aided surgical procedure and automate a flow through a series of user interface screens associated with a surgical procedure;

(4) providing the ability for a user to contact a probe against a portion of surgical instrument during a computer-aided surgical procedure and automate a flow through a series of user interface screens associated with a surgical procedure; and

(5) providing the ability for a user to manipulate a surgical instrument relative to a portion of a patient's body during a computer-aided surgical procedure and automate a flow through a series of user interface screens associated with a surgical procedure.

Other aspects, features and advantages of various aspects and embodiments of systems, methods, and apparatuses according to the invention are apparent from the other parts of this document.

### BRIEF DESCRIPTION OF THE DRAWINGS

FIG. 1 is an exemplary environment for a computer-aided surgical navigational system in accordance with an embodiment of the invention.

FIG. 2 is a surgical apparatus in accordance with an embodiment of the invention.

FIG. 3 is another surgical apparatus in accordance with an embodiment of the invention.

FIG. 4 is yet another surgical apparatus in accordance with an embodiment of the invention.

FIG. 5 is a flowchart for a method for using the computer-aided surgical navigational system shown in FIG. 1.

FIG. 6 is a flowchart for another method for using the computer-aided surgical navigational system according to another embodiment of the invention.

FIG. 7 is a flowchart for yet another method for using the computer-aided surgical navigational system according to another embodiment of the invention.

FIG. 8 is a flowchart for a surgical method used in conjunction with the computer-aided surgical navigational system shown in FIG. 1.

FIG. 9 is a flowchart for another surgical method used in conjunction with the computer-aided surgical navigational system according to another embodiment of the invention.

FIG. 10 is a flowchart for yet another surgical method used in conjunction with the computer-aided surgical navigational system according to another embodiment of the invention.

### DETAILED DESCRIPTION OF SPECIFIC EMBODIMENTS

Systems, methods, and apparatuses according to various embodiments of the invention address some or all of the above issues and combinations thereof. They do so by providing a computer-aided surgical system and methods to automatically provide a user interface associated with a surgical procedure for a user such as a surgeon or other surgical personnel. Such systems and methods are particularly useful for surgeons installing orthopedic components within a patient's body, wherein the computer-aided surgical navigation system can automatically display a user interface associated with a surgical procedure of interest when a particular surgical instrument, position of the instrument, or proximity or position of the instrument relative to a patient's body is detected or otherwise identified by the system.

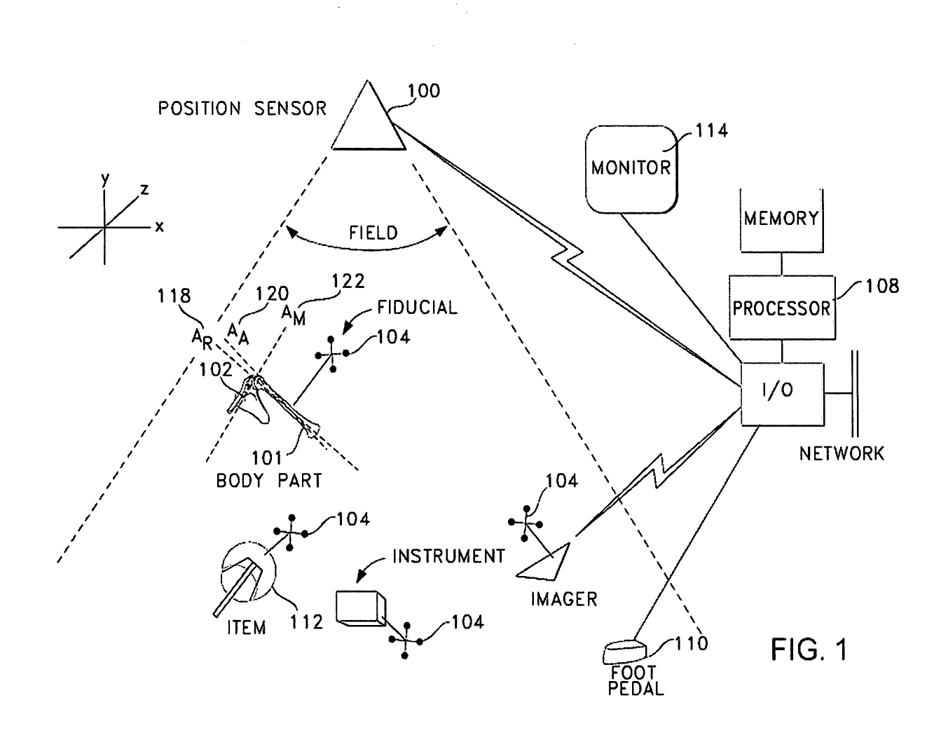

FIG. 1 is a schematic view showing an environment for using a computer-aided surgical navigation system according to some embodiments of the present invention, such as a surgery on a knee, in this case a knee arthroscopy. Systems and processes according to some embodiments of the invention can track various body parts such as a tibia 101 and femur 102 to which navigational sensors 100 may be implanted, attached or associated physically, virtually or otherwise.

Navigational sensors 100 may be used to determine and track the position of body parts, axes of body parts, implements, instrumentation, trial components and prosthetic components. Navigational sensors 100 may use infrared, electromagnetic, electrostatic, light sound, radio frequency or other desired techniques.

The navigational sensor 100 may be used to sense the position and orientation of navigational references 104 and therefore items with which they are associated. A navigational reference 104 can include fiducial markers, such as marker elements, capable of being sensed by a navigational sensor in a computer-aided surgical navigation system. The navigational sensor 100 may sense active or passive signals from the navigational references 104. The signals may be electrical, magnetic, electromagnetic, sound, physical, radio frequency, optical or visual, or other active or passive technique. For example in one embodiment, the navigational sensor 100 can visually detect the presence of a passive-type navigational reference. In an example of another embodiment, the navigational sensor 100 can receive an active signal provided by an active-type navigational reference. The surgical navigation system can store, process and/or output data relating to position and orientation of navigational references 104 and thus, items or body parts, such as 101 and 102 to which they are attached or associated.

In the embodiment shown in FIG. 1, computing functionality 108 such as one or more computer programs can include processing functionality, memory functionality, input/output functionality whether on a standalone or distributed basis, via any desired standard, architecture, interface and/or network topology. In one embodiment, computing functionality 108 can be connected to a display screen or monitor 114 on which graphics, data, and other user interfaces may be presented to a surgeon during surgery. The display screen or monitor 114 preferably has a tactile user interface so that the surgeon may point and click on the display screen or monitor 114 for tactile screen input in addition to or instead of, if desired, keyboard and mouse conventional interfaces.

Additionally, a foot pedal 110 or other convenient interface may be coupled to computing functionality 108 as can any other wireless or wireline interface to allow the surgeon, nurse or other user to control or direct functionality 108 in order to, among other things, capture position/orientation information when certain components are oriented or aligned properly. Items 112 such as trial components, instrumentation components may be tracked in position and orientation relative to body parts 101 and 102 using one or more navigational references 104.

The computing functionality 108 shown in FIG. 1 can also facilitate the display of one or more user interfaces via the display screen or monitor 114 in accordance with a desired surgical procedure. For example, one or more user interface pages or screens can be stored in memory associated with the computing functionality 108, and the pages can be organized or otherwise displayed in a predetermined order depending on a particular surgical procedure the user interface pages or screens are associated with. Suitable software capable of providing one or more user interface pages or screens is Achieve CAS Knee Version 2.0, distributed by Smith & Nephew of Memphis, Tenn. (United States). In one embodiment, user interface pages or screens with graphics, data, commands, or other information associated with a distal femoral cutting procedure can be stored and displayed when needed. In another embodiment, user interface pages or screens with graphics, data, commands, or other information associated with a proximal tibial cutting procedure can be stored and displayed when needed. In yet another embodiment, user interface pages or screens with graphics, data, commands, or other information associated with a femoral drilling procedure can be stored and displayed when needed. In any instance, user interface pages or screens with graphics, data, or other information associated with any surgical procedure or steps of a surgical procedure can be stored and displayed when needed.

Computing functionality 108 can, but need not, process, store and output on the display screen or monitor 114 various forms of data that correspond in whole or part to body parts 101 and 202 and other components for item 112. For example, body parts 101 and 102 can be shown in cross-section or at least various internal aspects of them such as bone canals and surface structure can be shown using fluoroscopic images. These images can be obtained using an imager 113, such as a C-arm attached to a navigational reference 104. The body parts, for example, tibia 101 and femur 102, can also have navigational references 104 attached. When fluoroscopy images are obtained using the C-arm with a navigational reference 104, a navigational sensor 100 “sees” and tracks the position of the fluoroscopy head as well as the positions and orientations of the tibia 101 and femur 102. The computer stores the fluoroscopic images with this position/orientation information, thus correlating position and orientation of the fluoroscopic image relative to the relevant body part or parts. Thus, when the tibia 101 and corresponding navigational reference 104 move, the computer automatically and correspondingly senses the new position of tibia 101 in space and can correspondingly move implements, instruments, references, trials and/or implants on the monitor 114 relative to the image of tibia 101. Similarly, the image of the body part can be moved, both the body part and such items may be moved, or the on-screen image otherwise presented to suit the preferences of the surgeon or others and carry out the imaging that is desired. Similarly, when an item 112, such as a stylus, cutting block, reamer, drill, saw, extramedullary rod, intramedullar rod, or any other type of item or instrument, that is being tracked moves, its image moves on monitor 114 so that the monitor 114 shows the item 112 in proper position and orientation on monitor 114 relative to the tibia 101. The item 112 can thus appear on the monitor 114 in proper or improper alignment with respect to the mechanical axis and other features of the tibia 101, as if the surgeon were able to see into the body in order to navigate and position item 112 properly.

The computing functionality 108 can also store data relating to configuration, size and other properties of items 112 such as joint replacement prostheses, implements, instrumentation, trial components, implant components and other items used in surgery. When those are introduced into the field of position/orientation sensor 100, computing functionality 108 can generate and display overlain or in combination with the fluoroscopic images of the body parts 101 and 102, computer generated images of joint replacement prostheses, implements, instrumentation components, trial components, implant components and other items 112 for navigation, positioning, assessment and other uses.

Instead of or in combination with fluoroscopic, MRI or other actual images of body parts, computing functionality 108 may store and output navigational or virtual construct data based on the sensed position and orientation of items in the surgical field, such as surgical instruments or position and orientation of body parts. For example, display screen or monitor 114 can output a resection plane, anatomical axis, mechanical axis, anterior/posterior reference plane, medial/lateral reference plane, rotational axis or any other navigational reference or information that may be useful or desired to conduct surgery. In the case of the reference plane, for example, display screen or monitor 114 can output a resection plane that corresponds to the resection plane defined by a cutting guide whose position and orientation is being tracked by navigational sensors 100. In other embodiments, display screen or monitor 114 can output a cutting track based on the sensed position and orientation of a reamer. Other virtual constructs can also be output on the display screen or monitor 114, and can be displayed with or without the relevant surgical instrument, based on the sensed position and orientation of any surgical instrument or other item in the surgical field to assist the surgeon or other user to plan some or all of the stages of the surgical procedure.

In some embodiments of the present invention, computing functionality 108 can output on the display screen or monitor 114 the projected position and orientation of an implant component or components based on the sensed position and orientation of one or more surgical instruments associated with one or more navigational references 104. For example, the system may track the position and orientation of a cutting block as it is navigated with respect to a portion of a body part that will be resected. Computing functionality 108 may calculate and output on the display screen or monitor 114 the projected placement of the implant in the body part based on the sensed position and orientation of the cutting block, in combination with, for example, the mechanical axis of the tibia and/or the knee, together with axes showing the anterior/posterior and medial/lateral planes. No fluoroscopic, MRI or other actual image of the body part is displayed in some embodiments, since some hold that such imaging is unnecessary and counterproductive in the context of computer aided surgery if relevant axis and/or other navigational information is displayed. Additionally, some systems use “morphed” images that change shape to fit data points or they use generic graphics or line art images with the data points displayed in a relatively accurate position or not displayed at all. If the surgeon or other user is dissatisfied with the projected placement of the implant, the surgeon may then reposition the cutting block to evaluate the effect on projected implant position and orientation.

The computer functionality 108 shown in FIG. 1 can also recognize certain surgical instruments or other objects by the navigational references 104 associated with the particular instruments. In one embodiment, this can be accomplished by storing information associated with a particular surgical instrument in memory of the computer functionality 108, and associating a discrete or unique navigational reference, such as 104, with the surgical instrument. The navigational reference, such as 104, can have a characteristic that can uniquely identify one navigational reference from another. A characteristic can include, but is not limited to, a shape, a size, a type, or a signal. Such characteristics can be stored by the computer functionality 108, and when the computer functionality 108 detects a particular previously stored characteristic for a navigational reference, such as 104, the computer functionality 108 can identify the surgical instrument associated with the navigational reference.

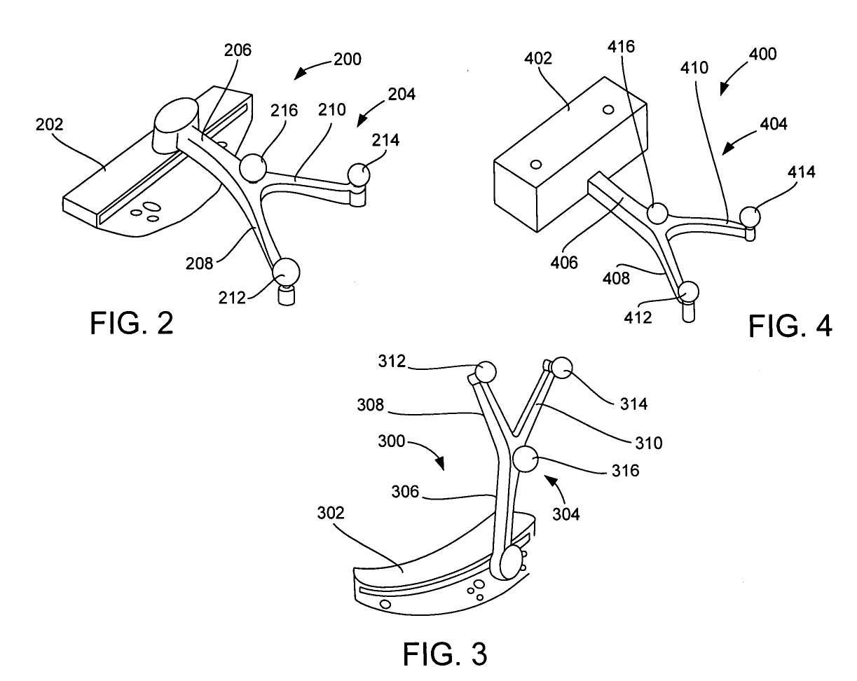

Examples of a characteristic, such as length, which can uniquely identify and distinguish between navigational references associated with respective surgical instruments are shown by reference to FIGS. 2-4. For example, as shown in FIG. 2, a navigational reference for a distal femoral guide can include a three-legged array and fiducials positioned adjacent to the ends of two legs, and a third fiducial positioned a central intersection of the three legs. The length of the two legs with fiducials can be a predetermined length, such as A millimeters. A navigational reference for a proximal tibial guide, as shown in FIG. 3, can also include a three-legged array and fiducials positioned adjacent to the ends of two legs, and a third fiducial positioned a central intersection of the three legs. The length of the two legs with fiducials can be a length different than the similar legs of the distal femoral guide, such as A+5 millimeters. Other navigational references, such as for a femoral four-in-one drill guide shown in FIG. 4, could also include a three-legged array, wherein the length of the two legs with fiducials can be a length different than the similar legs of the distal femoral guide and proximal tibial guide, such as A+10 millimeters. Arrays can also vary, for example, by different numbers of fiducials, different fiducail shapes, or otherwise be structurally different to be distinguishable from each other by the system. Other dimensions, shapes, configurations, or characteristics can be used to distinguish between navigational references. In this manner, the computer functionality 108 can distinguish between arrays or navigational references associated with respective surgical instruments.

The computer functionality 108 shown in FIG. 1 can also store associations between surgical instruments and surgical procedures. For example, a surgical instrument such as a distal femoral guide shown in FIG. 2 can be associated with one or more steps in a surgical procedure, such as a distal femoral cutting procedure. As explained above, each surgical procedure can be associated with one or more previously stored user interface pages or screens. Thus, when a surgical instrument is identified or otherwise detected by the computer functionality 108 via an associated array or navigational reference, such as 104, the computer functionality 108 can determine and identify a particular surgical procedure associated with the surgical instrument, and also determine and identify one or more previously stored user interface pages or screens associated with the surgical procedure. In this manner, a user can manipulate a surgical instrument in view of a computer-aided surgical system, as shown in FIG. 1, and the processing functionality can provide a series of user interface pages or screens in a predetermined order via a display screen or monitor, such as 114, depending on a particular surgical procedure the user interface pages or screens are associated with. As explained above, such user interface pages or screens can provide graphics, data, commands, or other information associated with a surgical procedure.

Additionally, computer functionality 108 can track any point in the navigational sensor 100 field such as by using a designator or a probe 116. The probe also can contain or be attached to a navigational reference 104. The surgeon, nurse, or other user touches the tip of probe 116 to a point such as a landmark on bone structure and actuates the foot pedal 110 or otherwise instructs the computer 108 to note the landmark position. The navigational sensor 100 “sees” the position and orientation of navigational reference 104 “knows” where the tip of probe 116 is relative to that navigational reference 104 and thus calculates and stores, and can display on the display screen or monitor 114 whenever desired and in whatever form or fashion or color, the point or other position designated by probe 116 when the foot pedal 110 is hit or other command is given. Thus, probe 116 can be used to designate landmarks on bone structure in order to allow the computer 108 to store and track, relative to movement of the navigational reference 104, virtual or logical information such as retroversion axis 118, anatomical axis 120 and mechanical axis 122 of femur 102, tibia 101 and other body parts in addition to any other virtual or actual construct or reference.

In one embodiment, contact of the probe 116 with a portion of an array or navigational reference, such as 104, can be detected via a sensor or position sensor 100 associated with the computer-aided surgical navigation system shown in FIG. 1. Using functionality described above, the computer functionality 108 can identify or otherwise determine a surgical instrument via the associated array or navigational reference 104. The computer functionality 108 can determine and identify a particular surgical procedure associated with the surgical instrument, and also determine and identify one or more previously stored user interface pages or screens associated with the surgical procedure. In this manner, a user can manipulate a probe and contact a portion of an array or navigational reference associated with a surgical instrument in view of a computer-aided surgical system, as shown in FIG. 1. The processing functionality can provide a series of user interface pages or screens in a predetermined order via a display screen or monitor, such as 114, depending on a particular surgical procedure the user interface pages or screens are associated with. As explained above, such user interface pages or screens can provide graphics, data, commands, or other information associated with a surgical procedure.

Systems and processes according to some embodiments of the present invention can communicate with suitable computer-aided surgical systems and processes such as the BrainLAB VectorVision system, the OrthoSoft Navitrack System, the Stryker Navigation system, the FluoroNav system provided by Medtronic Surgical Navigation Technologies, Inc. and software provided by Medtronic Sofamor Danek Technologies. Such systems or aspects of them are disclosed in U.S. Pat. Nos. 5,383,454; 5,871,445; 6,146,390; 6,165,81; 6,235,038 and 6,236,875, and related (under 35 U.S.C. Section 119 and/or 120) patents, which are all incorporated herein by this reference. Any other desired systems and processes can be used as mentioned above for imaging, storage of data, tracking of body parts and items and for other purposes.

These systems may require the use of reference frame type fiducials which have three or four, and in some cases five elements, tracked by sensors for position/orientation of the fiducials and thus of the body part, implement, instrumentation, trial component, implant component, or other device or structure being tracked. Such systems can also use at least one probe which the surgeon can use to select, designate, register, or otherwise make known to the system a point or points on the anatomy or other locations by placing the probe as appropriate and signaling or commanding the computer to note the location of, for instance, the tip of the probe. These systems also may, but are not required to, track position and orientation of a C-arm used to obtain fluoroscopic images of body parts to which fiducials have been attached for capturing and storage of fluoroscopic images keyed to position/orientation information as tracked by the sensors. Thus, the display screen or monitor can render fluoroscopic images of bones in combination with computer generated images of virtual constructs and references together with implements, instrumentation components, trial components, implant components and other items used in connection with surgery for navigation, resection of bone, assessment and other purposes.

In another embodiment, a portion of a patient's body can be associated with one or more arrays or navigational references, such as 104. The portion of the patient's body can be detected via a sensor or position sensor 100 associated with the computer-aided surgical navigation system shown in FIG. 1. As described above, a surgical instrument can also be identified or otherwise detected by the computer functionality 108 via an associated array or navigational reference, such as 104. Based on the position of the portion of the patient's body relative to the surgical instrument, both of which are detected or otherwise identified by the detection of associated arrays or navigational references, the computer functionality 108 can determine and identify a particular surgical procedure. In another embodiment, a surgical procedure can be selected or otherwise determined by the computer functionality 108 based on at least the proximity of the portion of the patient's body relative to the surgical instrument. The computer functionality 108 can then determine and identify one or more previously stored user interface pages or screens associated with the selected surgical procedure. In this manner, a user can manipulate a surgical instrument in relative to or in proximity with a portion of a patient's body in view of a computer-aided surgical system, as shown in FIG. 1. The computer functionality 108 can provide a series of user interface pages or screens in a predetermined order via a display screen or monitor, such as 114, depending on a particular surgical procedure the user interface pages or screens are associated with. As explained above, such user interface pages or screens can provide graphics, data, commands, or other information associated with a surgical procedure.

In yet another embodiment, the computer functionality 108 can provide data to permit navigation of a surgical instrument, orthopedic device, or item, such as 112, by a user performing a surgical procedure. Data can include, but is not limited to, text, graphics, a command, a screen display, or other information. For example, when a user, such as a surgeon, manipulates an item 112, the computer functionality 108 can receive position information associated with the item 112. The computer functionality 108 can process the position information, and can coordinate the position information with previously stored data, or with software programs or routines, to provide instructions or other direction to the user to navigate the item 112 relative to a patient's body or in a surgical procedure. In another embodiment, the computer functionality 108 can provide data for determining a surgical procedure. In this example, when a user, such as a surgeon, manipulates an item 112, the computer functionality 108 can receive position information associated with the item 112. The computer functionality 108 can utilize the position information with previously stored data, or with software programs or routines, to determine a surgical procedure associated with the item 112.

FIGS. 2-4 illustrate embodiments of a surgical apparatus in accordance with embodiments of the invention. Each of the apparatus shown in FIGS. 2-4 can be used in conjunction with the computer-aided surgical navigational system shown in FIG. 1. Furthermore, each of the apparatus shown in FIGS. 2-4 can be used in a surgical procedure, or in separate or overlapping steps of a surgical procedure, such as such as a knee arthroplasty. Other embodiments of surgical apparatus can exist in accordance with other embodiments of the invention.

In particular, FIG. 2 is a distal femoral guide and array apparatus in accordance with an embodiment of the invention. The distal femoral guide and array apparatus 200 can be a combination of a distal femoral guide 202 and an array or navigational reference 204. The array or navigational reference 204 shown in FIG. 2 includes a series of three legs 206, 208, 210 with fiducials 212, 214 positioned adjacent to the ends of two legs 208, 210, and a third fiducial 216 positioned adjacent to a central intersection of the three legs 206, 208, 210. The third leg 206 extends towards and mounts to a portion of the distal femoral guide 202.

FIG. 3 is a proximal tibial guide and array apparatus in accordance with an embodiment of the invention. The proximal tibial guide and array apparatus 300 can be a combination of a proximal tibial guide 302 and an array or navigational reference 304. The array or navigational reference 304 shown in FIG. 3 includes a series of three legs 306, 308, 310 with fiducials 312, 314 positioned adjacent to the ends of two legs 308, 310, and a third fiducial 316 positioned adjacent to a central intersection of the three legs 306, 308, 310. The third leg 306 extends towards and mounts to a portion of the proximal tibial guide 302.

FIG. 4 is a femoral four-in-one drill guide and array apparatus in accordance with an embodiment of the invention. The femoral four-in-one drill guide and array apparatus 400 can be a combination of a femoral four-in-one drill guide 402 and an array or navigational reference 404. The array or navigational reference 404 shown in FIG. 4 includes a series of three legs 406, 408, 410 with fiducials 412, 414 positioned adjacent to the ends of two legs 408, 410, and a third fiducial 416 positioned adjacent to a central intersection of the three legs 406, 408, 410. The third leg 406 extends towards and mounts to a portion of the femoral four-in-one drill guide 402.

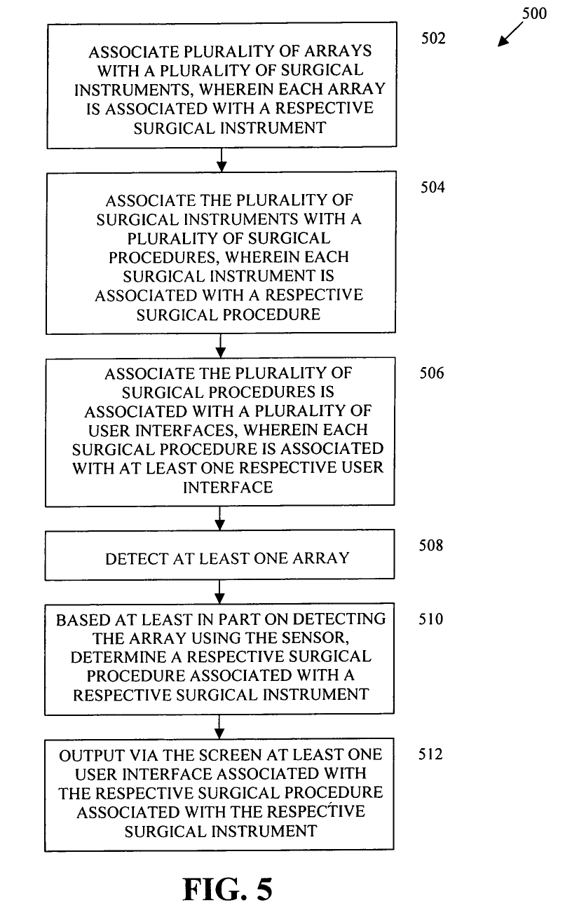

FIG. 5 illustrates a method performed by the computer-aided surgical navigational system shown in FIG. 1. The system, as described in FIG. 1, includes a display screen or monitor 114 and at least one sensor or position sensor 100. Other system embodiments can be used with the method 500 in accordance with other embodiments of the invention. Other method embodiments can have fewer or greater numbers of elements in accordance with other embodiments of the invention. The method 500 begins at block 502.

In block 502, a plurality of arrays is associated with a plurality of surgical instruments, wherein each array is associated with a respective surgical instrument. In the embodiment shown in FIG. 5, a processor such as 108 in FIG. 1, can store information associated with a plurality of arrays or navigational references, such as a characteristic of a navigational reference, for instance 104 in FIG. 1. Each respective array or navigational reference can then be associated with a respective surgical instrument, such as a distal femoral guide, proximal tibial guide, or a femoral four-in-one drill guide. This association information can be stored by the processor 108.

Block 502 is followed by block 504, in which the plurality of surgical instruments is associated with a plurality of surgical procedures, wherein each surgical instrument is associated with a respective surgical procedure. In the embodiment shown in FIG. 5, a processor such as 108 in FIG. 1, can store information associated with a plurality of surgical instruments, such as a distal femoral guide, proximal tibial guide, or a femoral four-in-one drill guide. Each surgical instrument can then be associated with a respective surgical procedure, such as a series of surgical steps. For instance, a surgical procedure can include, but is not limited to, a distal femoral cutting procedure, a proximal tibial cutting procedure, or a femoral four-in-one drilling procedure. This association information can be stored by the processor 108.

Block 504 is followed by block 506, in which the plurality of surgical procedures is associated with a plurality of user interfaces, wherein each surgical procedure is associated with at least one respective user interface. In the embodiment shown in FIG. 5, a processor such as 108 in FIG. 1, can store information associated with a plurality of surgical procedures, such as a distal femoral cutting procedure, proximal tibial cutting procedure, or a femoral four-in-one drilling procedure. The association information can include graphics, text, commands, or any other information stored or otherwise provided in a series of user interfaces capable of being displayed via a display screen or monitor, such as a 114 in FIG. 1.

Block 506 is followed by block 508, in which at least one array is detected. In the embodiment shown in FIG. 5, a sensor or position sensor, such as 100 in FIG. 1, can detect an array or navigational reference, such as 104, associated with a particular surgical instrument.

Block 508 is followed by block 510, in which based at least in part on detecting the array using the sensor, a respective surgical procedure associated with a respective surgical instrument is determined. In the embodiment shown in FIG. 5, the processor 108 can retrieve previously stored association information to determine or otherwise identify a particular surgical procedure based on the detection or identification of a respective array associated with a respective surgical instrument. For example, based on identification of a particular array or navigational reference, such as 104, associated with a distal femoral guide, the processor 108 can determine or otherwise identify a distal femoral cutting procedure or other series of surgical procedural steps.

Block 510 is followed by block 512, in which at least one user interface associated with the respective surgical procedure associated with the respective surgical instrument is output via the screen. In the embodiment shown in FIG. 5, the processor 108 can output via a display screen or monitor, such as 114, a user interface including graphics, text, or commands associated with the respective surgical procedure. For example, a processor 108 can display a series of user interfaces via a display screen or monitor 114 to collect and disseminate information associated with a distal femoral cutting procedure or other series of related surgical procedural steps.

The method 500 ends at block 512.

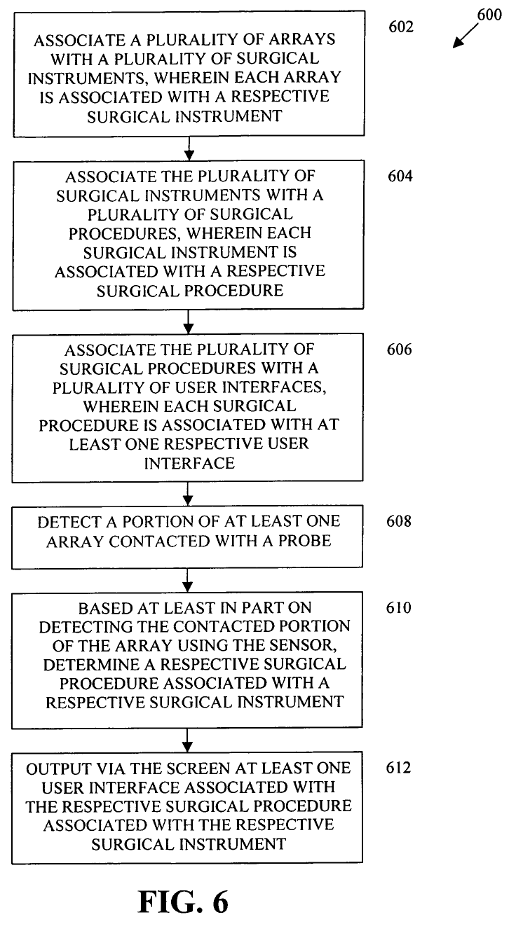

FIG. 6 illustrates another method performed by the computer-aided surgical navigational system shown in FIG. 1. The system, as described in FIG. 1, includes a display screen or monitor 114 and at least one sensor or position sensor 100. Other system embodiments can be used with the method 600 in accordance with other embodiments of the invention. Other method embodiments can have fewer or greater numbers of elements in accordance with other embodiments of the invention. The method 600 begins at block 602.

In block 602, a plurality of arrays is associated with a plurality of surgical instruments, wherein each array is associated with a respective surgical instrument. In the embodiment shown in FIG. 6, and similar to the embodiment described above in FIG. 5, a processor such as 108 in FIG. 1, can store information associated with a plurality of arrays or navigational references, such as a characteristic of a navigational reference, for instance 104 in FIG. 1. Each respective array or navigational reference can then be associated with a respective surgical instrument, such as a distal femoral guide, proximal tibial guide, or a femoral four-in-one drill guide. This association information can be stored by the processor 108.

Block 602 is followed by block 604, in which the plurality of surgical instruments is associated with a plurality of surgical procedures, wherein each surgical instrument is associated with a respective surgical procedure. In the embodiment shown in FIG. 6, and similar to the embodiment described above in FIG. 5, a processor such as 108 in FIG. 1, can store information associated with a plurality of surgical instruments, such as a distal femoral guide, proximal tibial guide, or a femoral four-in-one drill guide. Each surgical instrument can then be associated with a respective surgical procedure, such as a series of surgical steps. For instance, a surgical procedure can include, but is not limited to, a distal femoral cutting procedure, a proximal tibial cutting procedure, or a femoral four-in-one drilling procedure. This association information can be stored by the processor 108.

Block 604 is followed by block 606, in which the plurality of surgical procedures is associated with a plurality of user interfaces, wherein each surgical procedure is associated with at least one respective user interface. In the embodiment shown in FIG. 6, and similar to the embodiment described above in FIG. 5, a processor such as 108 in FIG. 1, can store information associated with a plurality of surgical procedures, such as a distal femoral cutting procedure, proximal tibial cutting procedure, or a femoral four-in-one drilling procedure. The association information can include graphics, text, commands, or any other information stored or otherwise provided in a series of user interfaces capable of being displayed via a display screen or monitor, such as a 114 in FIG. 1.

Block 606 is followed by block 608, in which a portion of at least one array contacted with a probe is detected. In the embodiment shown in FIG. 6, a sensor or position sensor, such as 100 in FIG. 1, can detect an array or navigational reference, such as 104, associated with the probe.

Block 608 is followed by block 610, in which based at least in part on detecting the contacted portion of the array using the sensor, a respective surgical procedure associated with a respective surgical instrument is determined. In the embodiment shown in FIG. 6, and similar to the embodiment described above in FIG. 5, the processor 108 can retrieve previously stored association information to determine or otherwise identify a particular surgical procedure based on the detection or identification of a respective array associated with a respective surgical instrument. For example, based on identification of the contacted portion of the particular array or navigational reference, such as 104, associated with a distal femoral guide, the processor 108 can determine or otherwise identify a distal femoral cutting procedure or other series of surgical procedural steps.

Block 610 is followed by block 612, in which at least one user interface associated with the respective surgical procedure associated with the respective surgical instrument is output via the screen. In the embodiment shown in FIG. 6, and similar to the embodiment described above in FIG. 5, the processor 108 can output via a display screen or monitor, such as 114, a user interface including graphics, text, or commands associated with the respective surgical procedure. For example, a processor 108 can display a series of user interfaces via a display screen or monitor 114 to collect and disseminate information associated with a distal femoral cutting procedure or other series of related surgical procedural steps.

The method 600 ends at block 612.

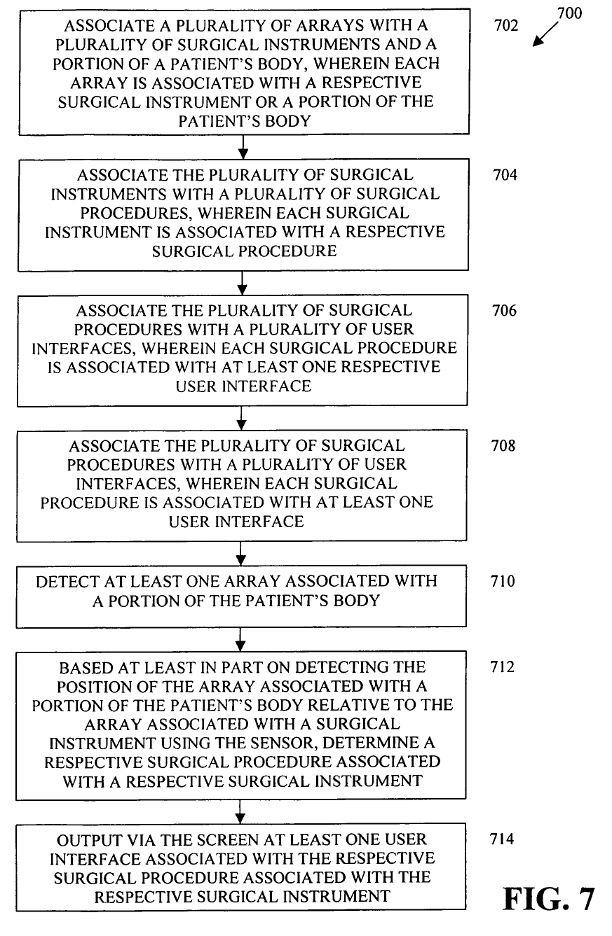

FIG. 7 illustrates yet another method performed by the computer-aided surgical navigational system shown in FIG. 1. The system, as described in FIG. 1, includes a display screen or monitor 114 and at least one sensor or position sensor 100. Other system embodiments can be used with the method 700 in accordance with other embodiments of the invention. Other method embodiments can have fewer or greater numbers of elements in accordance with other embodiments of the invention. The method 700 begins at block 702.

In block 702, a plurality of arrays is associated with a plurality of surgical instruments and a portion of a patient's body, wherein each array is associated with a respective surgical instrument or a portion of the patient's body. In the embodiment shown in FIG. 7, and similar to the embodiments described above in FIGS. 5 and 6, a processor such as 108 in FIG. 1, can store information associated with a plurality of arrays or navigational references, such as a characteristic of a navigational reference, for instance 104 in FIG. 1. One series of arrays or navigational references can be associated with a respective surgical instrument, such as a distal femoral guide, proximal tibial guide, or a femoral four-in-one drill guide. This association information can be stored by the processor 108. Another series of arrays or navigational references can be associated with a portion of a patient's body, such as a tibia or femur bone.

Block 702 is followed by block 704, in which the plurality of surgical instruments is associated with a plurality of surgical procedures, wherein each surgical instrument is associated with a respective surgical procedure. In the embodiment shown in FIG. 7, and similar to embodiments described above in FIGS. 5 and 6, a processor such as 108 in FIG. 1, can store information associated with a plurality of surgical instruments, such as a distal femoral guide, proximal tibial guide, or a femoral four-in-one drill guide. Each surgical instrument can then be associated with a respective surgical procedure, such as a series of surgical steps. For instance, a surgical procedure can include, but is not limited to, a distal femoral cutting procedure, a proximal tibial cutting procedure, or a femoral four-in-one drilling procedure. This association information can be stored by the processor 108.

Block 704 is followed by block 706, in which the plurality of surgical procedures is associated with a plurality of user interfaces, wherein each surgical procedure is associated with at least one user interface. In the embodiment shown in FIG. 7, and similar to the embodiments described above in FIGS. 5 and 6, a processor such as 108 in FIG. 1, can store information associated with a plurality of surgical procedures, such as a distal femoral cutting procedure, proximal tibial cutting procedure, or a femoral four-in-one drilling procedure. The association information can include graphics, text, commands, or any other information stored or otherwise provided in a series of user interfaces capable of being displayed via a display screen or monitor, such as a 114 in FIG. 1.

Block 706 is followed by block 708, in which at least one array associated with a portion of the patient's body is detected. In the embodiment shown in FIG. 7, a sensor or position sensor, such as 100 in FIG. 1, can detect an array or navigational reference, such as 104, associated with the portion of the patient's body.

Block 708 is followed by block 710, in which at least one array associated with a surgical instrument is detected. In the embodiment shown in FIG. 7, a sensor or position sensor, such as 100 in FIG. 1, can detect an array or navigational reference, such as 104, associated with the particular surgical instrument.

Block 710 is followed by block 712, in which based at least in part on detecting the position of the array associated with a portion of the patient's body relative to the array associated with a surgical instrument using the sensor, determining a respective surgical procedure associated with a respective surgical instrument. In the embodiment shown in FIG. 7, the processor 108 can retrieve previously stored association information to determine or otherwise identify a particular surgical procedure based on the detection or identification of the position of a respective array associated with a respective surgical instrument. For example, based on identification of a position of a particular array or navigational reference, such as 104, associated with a distal femoral guide, the processor 108 can determine or otherwise identify a distal femoral cutting procedure or other series of surgical procedural steps.

Block 712 is followed by block 714, in which at least one user interface associated with the respective surgical procedure associated with the respective surgical instrument is output via the screen. In the embodiment shown in FIG. 7, and similar to the embodiments described above in FIGS. 5 and 6, the processor 108 can output via a display screen or monitor, such as 114, a user interface including graphics, text, or commands associated with the respective surgical procedure. For example, a processor 108 can display a series of user interfaces via a display screen or monitor 114 to collect and disseminate information associated with a distal femoral cutting procedure or other series of related surgical procedural steps.

The method 700 ends at block 714.

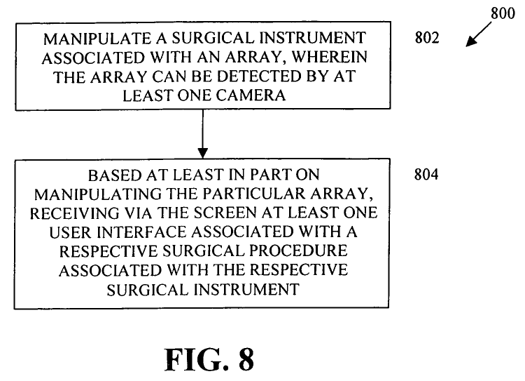

FIG. 8 illustrates a surgical method performed in conjunction with the computer-aided surgical navigational system shown in FIG. 1. The system, as described in FIG. 1, includes a display screen or monitor 114 and at least one sensor or position sensor 100. Other system embodiments can be used with the method 800 in accordance with other embodiments of the invention. Other method embodiments can have fewer or greater numbers of elements in accordance with other embodiments of the invention. The method 800 begins at block 802.

In block 802, a surgical instrument associated with an array is manipulated, wherein the array can be detected by the at least one sensor. In the embodiment shown in FIG. 8, a processor such as 108 in FIG. 1, can store information associated with a plurality of arrays or navigational references, such as a characteristic of a navigational reference, for instance 104 in FIG. 1. One or more arrays or navigational references can be associated with a respective surgical instrument, such as a distal femoral guide, proximal tibial guide, or a femoral four-in-one drill guide. This association information can be stored by the processor 108. When a user, such as a surgeon, uses a surgical instrument associated with an array in view of a sensor associated with a computer-aided surgical navigation system, such as in FIG. 1, the array can be detected by the sensor, and movement or other manipulation of the surgical instrument by the user can be detected by the sensor.

In one embodiment, a processor such as 108, can store information associated with a plurality of surgical procedures, such as a distal femoral cutting procedure, proximal tibial cutting procedure, or a femoral four-in-one drilling procedure. The association information can include graphics, text, commands, or any other information stored or otherwise provided in a series of user interfaces capable of being displayed via a display screen or monitor, such as a 114 in FIG. 1. Each respective surgical instrument can then be associated with a respective surgical procedure. The processor, such as 108, can store this information for subsequent retrieval and processing.

Block 802 is followed by block 804, in which based at least in part on manipulating the particular array, at least one user interface associated with a respective surgical procedure associated with the respective surgical instrument is received via the screen. In the embodiment shown in FIG. 8, the processor 108 can output via a display screen or monitor, such as 114, a user interface including graphics, text, or commands associated with the respective surgical procedure. For example, a processor 108 can display a series of user interfaces via a display screen or monitor 114 to collect and disseminate information associated with a distal femoral cutting procedure or other series of related surgical procedural steps.

The method 800 ends at block 804.

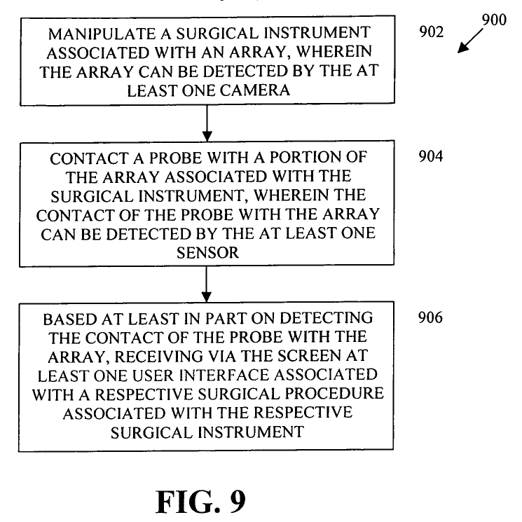

FIG. 9 illustrates another surgical method performed in conjunction with the computer-aided surgical navigational system shown in FIG. 1. The system, as described in FIG. 1, includes a display screen or monitor 114 and at least one sensor or position sensor 100. Other system embodiments can be used with the method 900 in accordance with other embodiments of the invention. Other method embodiments can have fewer or greater numbers of elements in accordance with other embodiments of the invention. The method 900 begins at block 902.

In block 902, a surgical instrument associated with an array is manipulated, wherein the array can be detected by the at least one sensor. In the embodiment shown in FIG. 9, and similar to the embodiment described above in FIG. 8, a processor such as 108 in FIG. 1, can store information associated with a plurality of arrays or navigational references, such as a characteristic of a navigational reference, for instance 104 in FIG. 1. One or more arrays or navigational references can be associated with a respective surgical instrument, such as a distal femoral guide, proximal tibial guide, or a femoral four-in-one drill guide. This association information can be stored by the processor 108. When a user, such as a surgeon, uses a surgical instrument associated with an array in view of a sensor associated with a computer-aided surgical navigation system, such as in FIG. 1, the array can be detected by the sensor, and movement or other manipulation of the surgical instrument by the user can be detected by the sensor.

In one embodiment, and similar to an embodiment described above in FIG. 8, a processor such as 108, can store information associated with a plurality of surgical procedures, such as a distal femoral cutting procedure, proximal tibial cutting procedure, or a femoral four-in-one drilling procedure. The association information can include graphics, text, commands, or any other information stored or otherwise provided in a series of user interfaces capable of being displayed via a display screen or monitor, such as a 114 in FIG. 1. Each respective surgical instrument can then be associated with a respective surgical procedure. The processor, such as 108, can store this information for subsequent retrieval and processing.

Block 902 is followed by block 904, in which a probe is contacted with a portion of the array associated with the surgical instrument, wherein the contact of the probe with the array can be detected by the at least one sensor. In the embodiment shown in FIG. 9, a sensor or position sensor, such as 100 in FIG. 1, can detect contact between the probe and an array or navigational reference, such as 104, associated with the portion of the patient's body.

Block 904 is followed by block 906, in which based at least in part on detecting the contact of the probe with the array, at least one user interface associated with a respective surgical procedure associated with the respective surgical instrument is received via the screen. In the embodiment shown in FIG. 9, and similar to the embodiment described in FIG. 8, the processor 108 can output via a display screen or monitor, such as 114, a user interface including graphics, text, or commands associated with the respective surgical procedure. For example, a processor 108 can display a series of user interfaces via a display screen or monitor 114 to collect and disseminate information associated with a distal femoral cutting procedure or other series of related surgical procedural steps.

The method 900 ends at block 906.

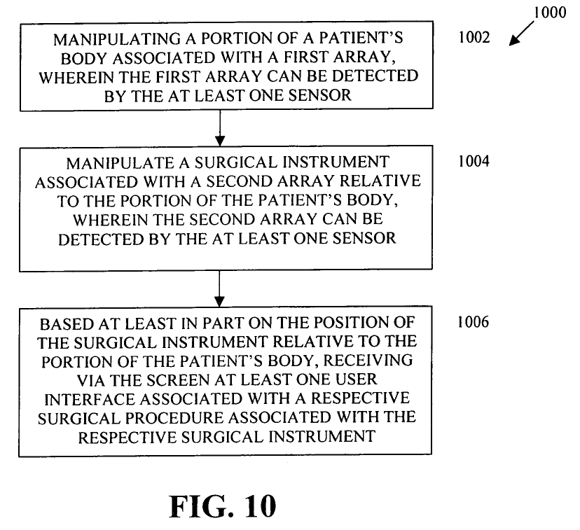

FIG. 10 illustrates yet another surgical method performed in conjunction with the computer-aided surgical navigational system shown in FIG. 1. The system, as described in FIG. 1, includes a display screen or monitor 114 and at least one sensor or position sensor 100. Other system embodiments can be used with the method 1000 in accordance with other embodiments of the invention. Other method embodiments can have fewer or greater numbers of elements in accordance with other embodiments of the invention. The method 1000 begins at block 1002.

In block 1002, manipulating a portion of a patient's body associated with a first array, wherein the first array can be detected by the at least one sensor. In the embodiment shown in FIG. 10, a processor such as 108 in FIG. 1, can store information associated with a plurality of arrays or navigational references, such as a characteristic of a navigational reference, for instance 104 in FIG. 1. One or more arrays or navigational references can be associated with a portion of a patient's body, such as a femur or tibia. This association information can be stored by the processor 108. When a user, such as a surgeon, moves or otherwise manipulates the portion of the patient's body associated with an array in view of a sensor associated with a computer-aided surgical navigation system, such as in FIG. 1, the array can be detected by the sensor, and movement or other manipulation of the portion of the patient's body by the user can be detected by the sensor.

In one embodiment, and similar to embodiments described above in FIGS. 8 and 9, a processor such as 108, can store information associated with a plurality of surgical procedures, such as a distal femoral cutting procedure, proximal tibial cutting procedure, or a femoral four-in-one drilling procedure. The association information can include graphics, text, commands, or any other information stored or otherwise provided in a series of user interfaces capable of being displayed via a display screen or monitor, such as a 114 in FIG. 1. Each respective surgical instrument can then be associated with a respective surgical procedure. The processor, such as 108, can store this information for subsequent retrieval and processing.

Block 1002 is followed by block 1004, in which a surgical instrument associated with a second array is manipulated relative to the portion of the patient's body, wherein the second array can be detected by the at least one sensor. One or more arrays or navigational references can be associated with a respective surgical instrument, such as a distal femoral guide, proximal tibial guide, or a femoral four-in-one drill guide. This association information can be stored by the processor 108. When a user, such as a surgeon, uses a surgical instrument associated with an array in view of a sensor associated with a computer-aided surgical navigation system, such as in FIG. 1, the array can be detected by the sensor, and movement or other manipulation of the surgical instrument relative to a portion of a patient's body by the user can be detected by the sensor.

Block 1004 is followed by block 1006, in which based at least in part on the position of the surgical instrument relative to the portion of the patient's body, at least one user interface associated with a respective surgical procedure associated with the respective surgical instrument is received via the screen. In the embodiment shown in FIG. 10, and similar to the embodiments described in FIGS. 8 and 9, the processor 108 can output via a display screen or monitor, such as 114, a user interface including graphics, text, or commands associated with the respective surgical procedure. For example, a processor 108 can display a series of user interfaces via a display screen or monitor 114 to collect and disseminate information associated with a distal femoral cutting procedure or other series of related surgical procedural steps.

The method 1000 ends at block 1006.

While the above description contains many specifics, these specifics should not be construed as limitations on the scope of the invention, but merely as exemplifications of the disclosed embodiments. Those skilled in the art will envision many other possible variations that within the scope of the invention as defined by the claims appended hereto.

The invention claimed is:

1. A computer-aided surgical navigational system with a display screen and at least one sensor, comprising:
a processor capable of
detecting a plurality of arrays, wherein each array is associated with a respective surgical instrument;
based at least in part on detecting at least one array using the sensor, determining a respective surgical procedure associated with the respective surgical instrument; and
outputting via the screen at least one user interface associated with the respective surgical procedure associated with the respective surgical instrument.

2. The system of claim 1, wherein a characteristic associated with the plurality of arrays can uniquely identify each array.

3. The system of claim 2, wherein the characteristic is selected from at least one of the following: shape, size, type, or signal.

4. The system of claim 1, wherein the plurality of arrays comprise at least one of the following: a fiducial member, a sensor, an infrared sensor, or a marker.

5. The system of claim 1, wherein the plurality of surgical instruments comprise at least one of the following: distal femoral cutting guide, a tibial cutting guide, a four-in-one drill guide, a cutting guide, a drill, a tool, an instrument used in an orthopedic surgical procedure, or an instrument used in a surgical procedure.

6. The system of claim 1, wherein the surgical procedure comprises at least one of the following: a distal femoral cutting procedure, a tibial cutting procedure, a cut, a series of cuts, a series of steps in a surgical procedure, a knee replacement procedure, or an orthopedic surgical procedure.

7. The system of claim 1, wherein the user interface comprises at least one of the following: a display of the surgical instrument relative to the patient's body, an instruction associated with the surgical procedure, a selection of measurements associated with the surgical procedure, or a command associated with the surgical procedure.

8. A method performed by a computer-aided surgical navigational system with a display screen and at least one sensor, comprising:
associating a plurality of arrays with a plurality of surgical instruments, wherein each array is associated with a respective surgical instrument;
associating the plurality of surgical instruments with a plurality of surgical procedures, wherein each surgical instrument is associated with a respective surgical procedure;
associating the plurality of surgical procedures with a plurality of user interfaces, wherein each surgical procedure is associated with at least one respective user interface;
detecting at least one array;
based at least in part on detecting the array using the sensor, determining a respective surgical procedure associated with a respective surgical instrument; and
outputting via the screen at least one user interface associated with the respective surgical procedure associated with the respective surgical instrument.

9. The method of claim 8, wherein a characteristic associated with the plurality of arrays can uniquely identify each array.

10. The method of claim 9, wherein the characteristic is selected from at least one of the following: shape, size, type, or signal.

11. The method of claim 8, wherein the plurality of arrays comprise at least one of the following: a fiducial member, a sensor, an infrared sensor, or a marker.

12. The method of claim 8, wherein the plurality of surgical instruments comprise at least one of the following: distal femoral cutting guide, a tibial cutting guide, a four-in-one drill guide, a cutting guide, a drill, a tool, an instrument used in an orthopedic surgical procedure, or an instrument used in a surgical procedure.

13. The method of claim 8, wherein the plurality of surgical procedures comprise at least one of the following: a distal femoral cutting procedure, a tibial cutting procedure, a cut, a series of cuts, a series of steps in a surgical procedure, a knee replacement procedure, or an orthopedic surgical procedure.

14. The method of claim 8, wherein the user interface comprises at least one of the following: a display of the surgical instrument relative to the patient's body, an instruction associated with the surgical procedure, a selection of measurements associated with the surgical procedure, or a command associated with the surgical procedure.

15. A computer-aided surgical navigational system with a display screen and at least one sensor, comprising:
a probe capable of
contacting a portion of a plurality of arrays associated with a plurality of surgical instruments, wherein each array is associated with a respective surgical instrument; and
a processor capable of
detecting the contacted portion of at least one array associated with a respective surgical instrument;
based at least in part on detection of the contacted portion of the array associated with the respective surgical instrument using the sensor, determining a respective surgical procedure associated with the respective surgical instrument; and
outputting via the screen at least one user interface associated with the respective surgical procedure associated with the respective surgical instrument.

16. The system of claim 15, wherein a characteristic associated with the plurality of arrays can uniquely identify each array.

17. The system of claim 16, wherein the characteristic is selected from at least one of the following: shape, size, type, or signal.

18. The system of claim 15, wherein the plurality of arrays comprise at least one of the following: a fiducial member, a sensor, an infrared sensor, or a marker.

19. The system of claim 15, wherein the plurality of surgical instruments comprise at least one of the following: distal femoral cutting guide, a tibial cutting guide, a four-in-one drill guide, a cutting guide, a drill, a tool, an instrument used in an orthopedic surgical procedure, or an instrument used in a surgical procedure.

20. The system of claim 15, wherein the surgical procedure comprises at least one of the following: a distal femoral cut, a distal femoral cutting procedure, a tibial cut, a tibial cutting procedure, a cut, a series of cuts, a series of steps in a surgical procedure, a knee replacement procedure, or an orthopedic surgical procedure.

21. The system of claim 15, wherein the user interface comprises at least one of the following: a display of the surgical instrument relative to the patient's body, an instruction associated with the surgical procedure, a selection of measurements associated with the surgical procedure, or a command associated with the surgical procedure.

22. A method performed by a computer-aided surgical navigational system with a display screen and at least one sensor, comprising:
associating a plurality of arrays with a plurality of surgical instruments, wherein each array is associated with a respective surgical instrument;
associating the plurality of surgical instruments with a plurality of surgical procedures, wherein each surgical instrument is associated with a respective surgical procedure;
associating the plurality of surgical procedures with a plurality of user interfaces, wherein each surgical procedure is associated with at least one respective user interface;
contacting a portion of at least one array with a probe;
detecting the contacted portion of the array;
based at least in part on detecting the contacted portion of the array using the sensor, determining a respective surgical procedure associated with a respective surgical instrument; and
outputting via the screen at least one user interface associated with the respective surgical procedure associated with the respective surgical instrument.

23. The method of claim 22, wherein a characteristic associated with the plurality of arrays can uniquely identify each array.

24. The system of claim 23, wherein the characteristic is selected from at least one of the following: shape, size, type, or signal.

25. The method of claim 22, wherein the plurality of arrays comprise at least one of the following: a fiducial member, a sensor, an infrared sensor, or a marker.

26. The method of claim 22, wherein the plurality of surgical instruments comprise at least one of the following: distal femoral cutting guide, a tibial cutting guide, a four-in-one drill guide, a cutting guide, a drill, a tool, an instrument used in an orthopedic surgical procedure, or an instrument used in a surgical procedure.

27. The method of claim 22, wherein the plurality of surgical procedures comprise at least one of the following: a distal femoral cutting procedure, a tibial cutting procedure, a cut, a series of cuts, a series of steps in a surgical procedure, a knee replacement procedure, or an orthopedic surgical procedure.

28. The method of claim 22, wherein the user interface comprises at least one of the following: a display of the surgical instrument relative to the patient's body, an instruction associated with the surgical procedure, a selection of measurements associated with the surgical procedure, or a command associated with the surgical procedure.

29. A computer-aided surgical navigational system with a display screen and a sensor, comprising:
a processor capable of
detecting a portion of a patient's body;
detecting a plurality of arrays associated with plurality of surgical instruments, wherein each array is associated with a respective surgical instrument;
determining a position of at least one array associated with a respective surgical instrument; and
based at least in part on determining the position of the array with respect to the portion of the patient's body using the sensor, determining a respective surgical procedure associated with the position of a particular array associated with the respective surgical instrument; and
outputting via the screen at least one user interface associated with a respective surgical procedure associated with the respective surgical instrument.

30. The system of claim 29, wherein a characteristic associated with the plurality of arrays can uniquely identify each array.

31. The system of claim 30, wherein the characteristic is selected from at least one of the following: shape, size, type, or signal.

32. The system of claim 29, wherein the plurality of arrays comprise at least one of the following: a fiducial member, a sensor, an infrared sensor, or a marker.

33. The system of claim 29, wherein the plurality of surgical instruments comprise at least one of the following: distal femoral cutting guide, a tibial cutting guide, a four-in-one drill guide, a cutting guide, a drill, a tool, an instrument used in an orthopedic surgical procedure, or an instrument used in a surgical procedure.

34. The system of claim 29, wherein the plurality of surgical procedures comprise at least one of the following: a distal femoral cut, a distal femoral cutting procedure, a tibial cut, a tibial cutting procedure, a cut, a series of cuts, a series of steps in a surgical procedure, a knee replacement procedure, or an orthopedic surgical procedure.

35. The system of claim 29, wherein the user interface comprises at least one of the following: a display of the surgical instrument relative to the patient's body, an instruction associated with the surgical procedure, a selection of measurements associated with the surgical procedure, or a command associated with the surgical procedure.

36. A method performed by a computer-aided surgical navigational system with a display screen and at least one sensor, comprising:
associating a plurality of arrays with a plurality of surgical instruments and a portion of a patient's body, wherein each array is associated with a respective surgical instrument or a portion of the patient's body;
associating the plurality of surgical instruments with a plurality of surgical procedures, wherein each surgical instrument is associated with a respective surgical procedure;
associating the plurality of surgical procedures with a plurality of user interfaces, wherein each surgical procedure is associated with at least one user interface;
detecting at least one array associated with a portion of the patient's body;
detecting at least one array associated with a surgical instrument;
based at least in part on detecting the position of the array associated with a portion of the patient's body relative to the array associated with a surgical instrument using the sensor, determining a respective surgical procedure associated with a respective surgical instrument; and
outputting via the screen at least one user interface associated with the respective surgical procedure associated with the respective surgical instrument.

37. The method of claim 36, wherein a characteristic associated with the plurality of arrays can uniquely identify each array.

38. The method of claim 37, wherein the characteristic is selected from at least one of the following: shape, size, type, or signal.

39. The method of claim 36, wherein the plurality of arrays comprise at least one of the following: a fiducial member, a sensor, an infrared sensor, or a marker.

40. The method of claim 36, wherein the plurality of surgical instruments comprise at least one of the following: distal femoral cutting guide, a tibial cutting guide, a four-in-one drill guide, a cutting guide, a drill, a tool, an instrument used in an orthopedic surgical procedure, or an instrument used in a surgical procedure.

41. The method of claim 36, wherein the plurality of surgical procedures comprise at least one of the following: a distal femoral cutting procedure, a tibial cutting procedure, a cut, a series of cuts, a series of steps in a surgical procedure, a knee replacement procedure, or an orthopedic surgical procedure.

42. The method of claim 36, wherein the user interface comprises at least one of the following: a display of the surgical instrument relative to the patient's body, an instruction associated with the surgical procedure, a selection of measurements associated with the surgical procedure, or a command associated with the surgical procedure.

43. A surgical method performed in conjunction with a computer-aided surgical navigational system with a display screen and at least one sensor, comprising:
manipulating a surgical instrument associated with an array, wherein the array can be detected by the at least one sensor; and
based at least in part on manipulating the particular array, receiving via the screen at least one user interface associated with a respective surgical procedure associated with the respective surgical instrument.

44. A surgical method performed in conjunction with a computer-aided surgical navigational system with a display screen and at least one sensor, comprising:
manipulating a surgical instrument associated with an array, wherein the array can be detected by the at least one sensor;
contacting a probe with a portion of the array, wherein the contact of the probe with the array can be detected by the at least one sensor; and
based at least in part on detecting the contact of the probe with the array, receiving via the screen at least one user interface associated with a respective surgical procedure associated with the respective surgical instrument.

45. A surgical method performed in conjunction with a computer-aided surgical navigational system with a display screen and at least one sensor, comprising:
manipulating a portion of a patient's body associated with a first array, wherein the first array can be detected by the at least one sensor;
manipulating a surgical instrument associated with a second array relative to the portion of the patient's body, wherein the second array can be detected by the at least one sensor; and
based at least in part on the position of the surgical instrument relative to the portion of the patient's body, receiving via the screen at least one user interface associated with a respective surgical procedure associated with the respective surgical instrument.
